(19) United States
(12) Patent Application Publication
(10) Pub. No.: US 2002/0115095 A1
(43) Pub. Date: Aug. 22, 2002

(54) Production method of micro-reactors gene chips

(75) Inventor: Ping Zhu, Beijing (CN)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: Peking University, CHINA

(21) Appl. No.: 10/043,995

(22) Filed: Jan. 11, 2002

(30) Foreign Application Priority Data
Feb. 21, 2001 (CN) 01104063.7

(51) Int. Cl. C12Q 1/68; C12M 1/34

(52) U.S. Cl. 435/6; 435/287.2

(57) ABSTRACT

This invention is the procedure of a kind of gene chips. Large amounts of PCR reactions can be performed in a small chip. 100-20,000 Micro-Reactors (MRs) will be implemented in the chips, in which a set of reactive media is immobilized. Many kinds of PCR reactions will be performed simultaneously with the same sample; the selected hundreds of gene will be amplified millions folds, then the several kinds of gene variance can be identified.

### CROSS-REFERENCE TO RELATED APPLICATIONS

This application claims priority to Chinese application No. 01104063.7 filed Feb. 21, 2001.

### BACKGROUND OF THE INVENTION

The present invention relates to gene chips and polymerase chain reaction (PCR). It can be applied to identify and analyze gene variants in biomedicine. Multifarious genes of human beings, animals and plants can be studied simultaneously, more than 100 kinds of gene variants such as gene mutation, gene deletion or gene rearrangement can be detected in just once reaction.

Gene chips, or calls microarrays, usually mean to immobilize thousands of oligonucleotide or another gene probes in microfabricated patterns on the surface of a solid support for analyzing the expression of genes in a certain life beings. U.S. Pat. No. 5,837,832 described methods for making this kind of gene chips. Based on this method, some gene chips that detect genes of microorganisms or virus and reveal the expression of the genes of cell lines have presented. All of the gene chips heretofore are based on theory of nucleic acid hybridization, see Chinese Patent application Ser. No. 99114460 and 98120104. However the principle of this technology is suited to detect expression of genes, detect the gene mutation, deletion or rearrangement of genomic DNA, to which related to most of the heredity variances and phenotype changes in life beings, is quite difficulty by these chips. Especially in clinic medicine, the gene mutation, deletion or rearrangement rather than gene expression are of clinical significance in-patients with carcinoma or leukemia. The question left is how to distinguish these gene variances.

PCR, a method to amplify mass of a specific gene in vitro, has been widely applied in biomedical field. Appropriate PCR primers will help to detect gene mutation directly with template from a single cell or its genomic DNA. The fusion gene that generated from gene rearrangement can be detected by analysis of presence of the PCR products. The deletion or insertion of a gene can be identified by analysis of the length of the PCR products. The mutation of a certain gene can be detected by modification of the 3′ end of the primers with the mutation site, the existence of the mutation can be inferred by the positive results of the PCR products. Analysis of the change of the PCR products is an efficient method to evaluate the gene rearrangement, mutation and deletion of the genomic DNA from an organism. But the number of the gene in a live being is staggering that of human beings is about 30,000-50,000. When there is a genetic variation, these gene variances are different, even in the organism with the same phenotype, different gene variance exits in different phase of life. Take the gene mutation in leukemia, which I studied in recent years for example. There are at least 90 kinds of typical translocations that are specific in classification, fusion genes are generated by the gene rearrangement after the translocation. Detection of fusion gene by PCR is a sensitive and effective method to identify different leukemia. However the existence of a certain fusion gene should be known before the corresponding PCR test being chosen. In case of the he existence of the certain fusion gene can not be predicted, many times of different PCR must be done for just one patient. Mass time, efforts and money will be cost but the results are not always accurate. The clinical application of this method is quite difficult. To tackle this problem, I created a method named multi-step nested RT PCR (see Chin J Pedia, 2001), with primers coming from 29 chromosomal translocation broken points in leukemia (including more than 80 broken points of mRNA or splicing variants), to amplify simultaneously several genes in a tube. A sample can be divided into 8 tubes, and then 29 fusion genes can be detected at one time. Although the rate of success of this method is higher than other methods, the multifarious genes related with leukemia still can not be evaluated in one time.

### BRIEF SUMMARY OF THE INVENTION

The objective of this invention is to create a gene chip by which the great number of suspected variant genes in an organism could be evaluated in one time. The principle of this chip is based on PCR rather than molecular hybridization; thus the sensitivity is higher than the traditional chips. In the chip there are hundreds of or more MRs, where PCR reaction proceeds. Genes in MRs will be amplified millions folds or more, thus the shortcoming of the conventional PCR not able to amplify large number of genes will be overcome.

### DETAILED DESCRIPTION OF THE INVENTION

The manipulation of the gene chip is simple. First a little number of cells will be degenerated in reactive solution, or dilute the genomic DNA by reactive solution, then add into the chip through sample hole. The reaction will last for about 1 hour, and then the results of detection of the hundreds of genes will be displayed on the screen of the computer.

The volume of the PCR chips is less than 3×2×0.2 cm, 100-20,000 Micro-Reactors(MRs) will be established on the same surface. The MRs are connected by a set of pipeline, through which a sample can flow into all the MRs. A set of reactive media exits in each of the MR, including primers to amplify different gene and gene mutation, fluorescent probe, and the shared dNTP, DNA polymerase, Mg²+ and KCl etc. 100-20,0000 reactions can be performed simultaneously; the mutation, deletion or rearrangement of more than 100 genes can be detected at one time.

To ensure the PCR to proceed smoothly and reduce the cost of the chips to level of single use, the chips should be made from such materials as silica or plastic which is able to endure the temperature of 0-99° C. for up to 24 hours. All the reactions are performed in the enclosed single use chips, thus the false positive results due to the contamination of the former PCR products would be avoided.

The volume of the MRs will be less than 1 microliter and the quantity of the reactive media will be tiny. To facilitate the inside reaction, production in batch and the carriage of the chips, the reactive media will be immobilized on the bottom of the MRs. The appropriate reactive buffer adsorbed by nanometer level magnetic beads will be added into the MRs by machine hands. A certain quantity of the reactive media will be lyophilized and immobilized on the bottom of the MRs by exogenous magnetic force from specific detector when samples are infused in case of the media being washed away. Magnetic stirring apparatus will be employed to accelerate the reaction. To simplify the PCR reaction, the polymerase in the reactive media is heat-activated; the efficiency of the PCR is higher than the conventional ones because all the reaction will be fulfilled at one time.

The PCR primers are constituted by different specific gene fragments, the number of the primes can reach to hundreds or higher. The corresponding primers will analyze large amount of variation genes. The presence of fusion gene generated from gene rearrangement can be detected by analysis of the PCR products. The deletion or insertion of a gene can be identified by analysis of the length of the PCR products. The mutation of a certain gene can be detected by modification of the 3′ end of the primers with the mutation site; the existence of the mutation can be inferred by the positive results of the PCR products. Analysis of the change of the PCR products by corresponding primers is an efficient method to evaluate the gene rearrangement, mutation and deletion of the genomic DNA.

The greatest problem in PCR is quantitative analysis. Quantitative analysis is important for the identification by gene analysis of the few variant cells from the great number of normal cells, especially the ounce of carcinoma cells in-patients with tumors or leukemia. The conventional quantitative analysis of PCR is to speculate the amount of the DNA from variant/carcinoma cells by the amount of the product at the end of the reaction, with genes stable expressed e.g. β-actin as internal reference. The main flaw of this method is not able to overcome the platform effect of PCR. The superiority of fluorescence quantitative analysis is obvious. A fragment of gene is commonly labeled by fluorescent dyes to sever as specific probe, the will integrate into the PCR product during the reaction. The amount of the DNA template will be learnt by the analysis of the optical density of the fluorescence in the PCR product, the number of the variant/carcinoma cells will further be figured out. The analysis will be continuous during the reaction, making it inferior to analyze just at the end of the reaction. This kind of real time monitoring overcome the platform effect in the later period of the PCR reaction where the DNA can not any more be amplified by folds. The top of the MRs in the PCR chips is transparent, through which the optical density of the fluorescence in the MRs will be monitored.

There is a kind of fluorescent probe, which is labeled by a pair of fluorescent dyes. Fluorescence can not be detected due to an interactive mechanism called “FRET (fluorescence remit energy transformation)” between the two dyes. Polymerase will digest the inhibitor of fluorescence on one end when the probe combines the PCR product, then the fluorescence on the other end of the probe turns into detectable and the PCR product can be distinguished.

The fluorescent probes can be replaced by that labeled by other chromogenic agents. The optical density of the fluorescence or other dyes in the MRs will indicate the amount of the PCR product. The detection of fluorescence in a MR indicates the presence of the corresponding specific gene. The top surface of the MRs in the PCR chip is transparent, through which the fluorescence can be excited and detected. The optical density of the fluorescence in a MR will be can be excited by laser ND and detected by the image collector per minute. The PCR products present when the fluorescence in a MR becomes detectable. The image analysis system collects the information of fluorescence and transfers it into a computer to process. The quantitative analysis based on the data of optical density of each MR will be continuous during the entire reaction. A reference system about 20 MRs will be built in the chip, the amounts of the DNA template in these MRs are known. The standard curve of the DNA amount will be set based on the data collected from these reference MRs, the quantitative analysis of the other MRs is all based on this standard curve. Probes labeled with other chromogenic agents can replace the fluorescent probes.

The superiority of my invention is obvious too. The volume of this chip is not large than the conventional ones and larger amount of gene can also be analyzed at one time, but the sensitivity and possibility to detect a suspected mutation is higher. This chip overcomes the designed flaws of the other chips. The principle of this chip is based on PCR rather than molecular hybridization; thus the sensitivity is higher than the traditional chips. PCR gene chips broaden the field of application of gene chips in Biomedicine, this chips suit to identify the variant gene rearrangement, deletion or mutation in human beings, animals or vegetables. A few degenerated cells or genomic DNA samples are enough to detect hundreds of gene variance in the samples. PCR reactions are performed in the hundreds or more “micro-labs” i.e. MRs; the variant genes in little amounts of cell will be amplified up to millions folds. The reactive buffer, fluorescent probes and heat-activated polymerase of the PCR reaction will be adsorbed by nanometer level magnetic beads and will be immobilized on the bottom of the MRs. These chips can be used in the diagnosis of carcinomas and leukemia in clinics, analysis of human genome; identification of polymorphism of genes or predisposition of disease; gene diagnosis of hereditary disorders, screening of variant animal or plant genes and the identification of pathogenic microorganism.

We claim:

1. A gene chip, characterized by 100-20,000 Micro-Reactors (MRs) on the same plane; Polymerase Chain Reaction (PCR) can be performed in the MRs. The MRs are connected by a set of pipeline, through which a sample can flow into all the MRs. A set of reactive media exits in each of the MR, then PCR can be performed simultaneously under the same condition. Real-time quantification can be carried out by detecting the change of the optical density in the MR through its top surface, which is transparent.

2. In all the MRs of the chip of claim 1, PCR can be performed simultaneously, after the infusion of a little amount of genomic DNA or degenerative cells from human beings, animals or plants into the MR. Therefore more than 100 kinds of gene variants such as gene mutation, gene deletion or gene rearrangement can be detected in just oncereaction.

3. The chip of claim 1, wherein said material is silica or plastic which is able to endure the temperature of 0-99° C. for up to 24 hours.

4. The chip of claim 1, wherein said agents in the reactive media are adsorbed by magnetic beads whose diameters are in nanometer level. The said micro-beads will be immobilized on the bottom of the MRs by exogenous magnetic force when samples are infused. Magnetic stirring apparatus will be employed to accelerate the reaction.

5. The chip of claim 1, wherein said PCR primers in the reactive media are made up with primers to amplify multifarious functional genes, immobilized on the bottom of the MRs after being mixed.

6. The reactive media of claim 1 includes a kind of fluorescent probe, which is labeled by a pair of fluorescent dyes. Fluorescence can not be detected due to an interactive mechanism called “FRET (fluorescence remit energy transformation)” between the two dyes. Polymerase will digest the inhibitor of fluorescence on one end when the probe combines the PCR product, then the fluorescence on the other end of the probe turns into detectable and the PCR product can be distinguished.

7. The fluorescent probes of claim 1 can be replaced by probes labeled with other chromogenic agents.

8. The top surface of the MRs of claim 1 is transparent. The optical density of the fluorescence or other dyes in the MRs can be detected through the said surface. The optical density of the dyes is in proportion to the amount of the PCR product. Image collector will monitor the real time change of the optical density and transfer the data collected into the image analysis system of the computer software.

9. The reactive media of claim 1, wherein said polymerase is heat-activated, the efficiency of the PCR is higher than that of PCR using common polymerase.
